(19) United States
(12) Patent Application Publication
(10) Pub. No.: US 2006/0206007 A1
(43) Pub. Date: Sep. 14, 2006

(54) Disposable illuminator endoscope

(76) Inventor: John L. Bala, Pomfret Center, CT (US)

Correspondence Address: ALIX YALE & RISTAS LLP, 750 MAIN STREET, SUITE 1400, HARTFORD, CT, 06103, US

(21) Appl. No.: 11/079,724

(22) Filed: Mar. 14, 2005

(51) Int. Cl. A61B 1/06; A61B 1/04

(52) U.S. Cl. 600/182; 600/160; 600/178; 600/121

(57) ABSTRACT

A disposable illuminator endoscope includes a reusable endoscopic device and a disposable illuminator. The endoscopic device includes an optical image pathway, an operator assembly mounted to the optical image pathway proximal end, a fiber optic annulus surrounding a proximal end portion of the optical image pathway, and a light source optically coupled to the fiber optic annulus. The illuminator includes a light path, a sealing lens mounted to the light path distal end, and a protective sleeve having a distal end portion mounted to a proximal end portion of the light path. The disposable illuminator is mounted on the reusable endoscopic device by inserting the optical image pathway into the light path and placing the protective sleeve around the operator assembly, such that the distal end of the optical image pathway is disposed proximate to the sealing lens and the light path proximal end interfaces with fiber optic annulus to optically couple the disposable illuminator to the light source. The disposable illuminator is removed from the reusable endoscopic device by withdrawing the optical image pathway and the operator assembly from the light path and the protective sleeve, respectively.

### BACKGROUND OF THE INVENTION

This invention relates generally to endoscopes which are used in the field of medicine. More particularly, the present invention relates to endoscopes having disposable components.

Originally developed in 1966 by Dr. H. H. Hopkins, the rigid endoscope has changed little in basic form and design, generally having a diameter of 10 mm and a length of 350-400 mm. The Hopkins design included non-imaging fiberoptics for illumination conduits and optics having a telescopic design that is focused at infinity, thus enabling the endoscope to remain in focus throughout its operating range. An objective lens captures the image from inside the body and relays it through a series of rods and lenses to convey the image from the objective elements to an eyepiece at the end of the endoscope. The system operates in a manner such that as light exits one rod element, a collimating lens intercepts and re-directs the optical rays to a second rod, and, then, to a third rod lens and so forth until the image has been conveyed to an eyepiece at the end of the device. Additional lenses located in the eyepiece capture the image from the final relay system and focus it on the human eye.

Such “rigid” scopes are used to perform procedures within the trunk of the body. Rigid surgical endoscopes enter the body through relatively large incisions made by a trocar or a cannula. Pelvic examinations, tubal ligation, and gall bladder removals were among the first surgical procedures using endoscopes to be approved by the FDA, and remain among the most often performed surgeries. Due to the potential for bleeding, pain and trauma, procedures utilizing rigid endoscopes are preformed in a hospital or clinic and generally necessitate either local or general anesthesia. Accordingly, the entire process is both traumatic and expensive for the patient.

A smaller version of the Hopkins design, an arthroscope, is 2.7 mm in diameter and 150-200 mm in length. The principles of imaging are the same, only the applications are different. Because smaller diameters minimize trauma to human tissue, orthopedic specialists currently use these smaller diameter scopes to diagnose and treat injuries. More than 2.7 million arthroscopic procedures are performed annually. These include over 1.7 million knee procedures, 500,000 shoulder injuries, 200,000 elbow, ankle and wrist injuries, and 200,000 spinal procedures.

Much of the early development in medical fiber optics for flexible endoscopes was performed at the American Optical Corporation (AOC). AOC pioneered the combining of thousands of individual, spatially aligned fibers that are needed to relay an image along the length of the fiber. AOC's early research created fiber optic illumination systems, coherent fiber imaging and remote articulation technologies that are still the basis of all modern flexible endoscopes. Flexible endoscopes require objective lenses with optical designs that are different for each fiber diameter, field of view, or working distance. These designs are optimized for a given application along with the illumination conduits. The imaging fibers are equivalent to the rod lenses in the rigid scope, but are capable of conveying the image over much longer lengths without significant transmission loss. However, as a general rule performance of the fiber optics increases with diameter and decreases with length while versatility of the fiber optics decreases with diameter and increases with length.

Both rigid and flexible endoscopes require the use of illumination conduits to provide light for illuminating the interior of the patient's body. From a practical and functional perspective, glass fiber is generally the best material for such use. Fiber optic light guides can transmit light frequencies from the ultra violet to the near infrared spectrum. They are relatively non-expensive, durable, optically efficient and functional. Glass is also able to withstand frequent sterilization, by autoclaving and other standard methods, required to utilize conventional endoscopes for more than one patient.

### SUMMARY OF THE INVENTION

Briefly stated, the invention in a preferred form is a disposable illuminator endoscope which comprises a reusable endoscopic device and a disposable illuminator. The endoscopic device includes an optical image pathway, an operator assembly mounted to the optical image pathway proximal end, a fiber optic annulus surrounding a proximal end portion of the optical image pathway, and a light source optically coupled to the fiber optic annulus. The illuminator includes a light path, a sealing lens mounted to the light path distal end, and a protective sleeve having a distal end portion mounted to a proximal end portion of the light path. The disposable illuminator may be mounted on the reusable endoscopic device by inserting the optical image pathway into the light path and placing the protective sleeve around the operator assembly, such that the distal end of the optical image pathway is disposed proximate to the sealing lens and the light path proximal end interfaces with fiber optic annulus to optically couple the disposable illuminator to the light source. The disposable illuminator may be removed from the reusable endoscopic device by withdrawing the optical image pathway and the operator assembly from the light path and the protective sleeve, respectively.

Preferably, the optical image pathway comprises a flexible, coherent fiber optic imaging bundle or probe, and the light path is composed of a flexible material.

The optical image pathway may be covered with a metal sheath that precludes infiltration of light into the optical image pathway. Preferably, the metal sheath is flexible.

The fiber optic annulus includes a circular array of interface fiber optics forming a light annulus around the optical image pathway proximal end portion.

The operator assembly preferably includes a CCD camera or CCD sensor array and an imaging lens assembly positioned between the proximal end of the optical image pathway and the CCD camera or CCD sensor array. The operator assembly may also include a focus adjustment disposed between the imaging lens assembly and the CCD camera or CCD sensor array. Further, the operator assembly may include at least one objective lens.

It is an object of the invention to provide a new and improved endoscope system for providing medical imaging.

It is also an object of the invention to provide a new and improved endoscope system having a disposable illuminator forming the outer envelope of the end oscope.

Other objects and advantages of the invention will become apparent from the drawings and specification.

### BRIEF DESCRIPTION OF THE DRAWINGS

The present invention may be better understood and its numerous objects and advantages will become apparent to those skilled in the art by reference to the accompanying drawings in which:

FIG. 1 is a top view of a disposable illuminator endoscope in accordance with the invention, showing the disposable illuminator completely installed on the reusable endoscopic device;

FIG. 2 is a side view of the disposable illuminator endoscope of FIG. 1, showing the disposable illuminator partially installed on the reusable endoscopic device;

FIG. 3 is a side view of the disposable illuminator of FIG. 1;

FIG. 4 is a schematic view of the reusable endoscopic device of FIG. 1;

FIG. 5 is a cross-section taken along line 5-5 of FIG. 1; and

FIG. 6 is a simplified perspective view of an endoscopic imaging system having a disposable illuminator endoscope in accordance with the present invention.

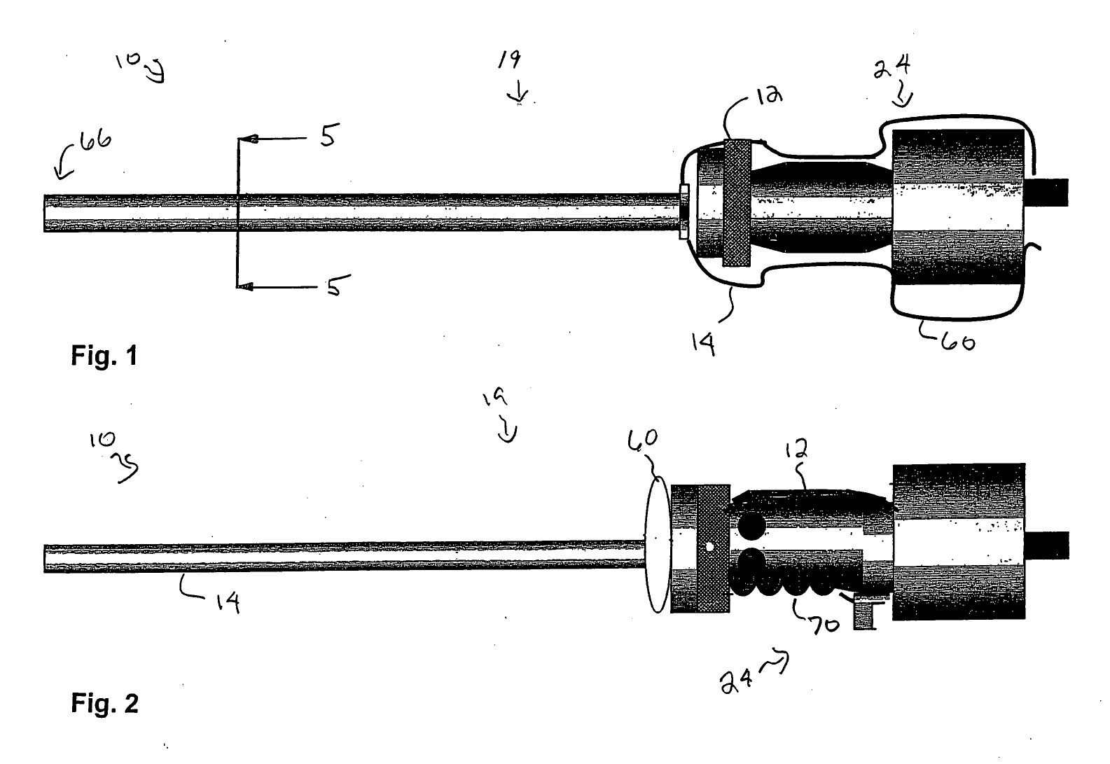

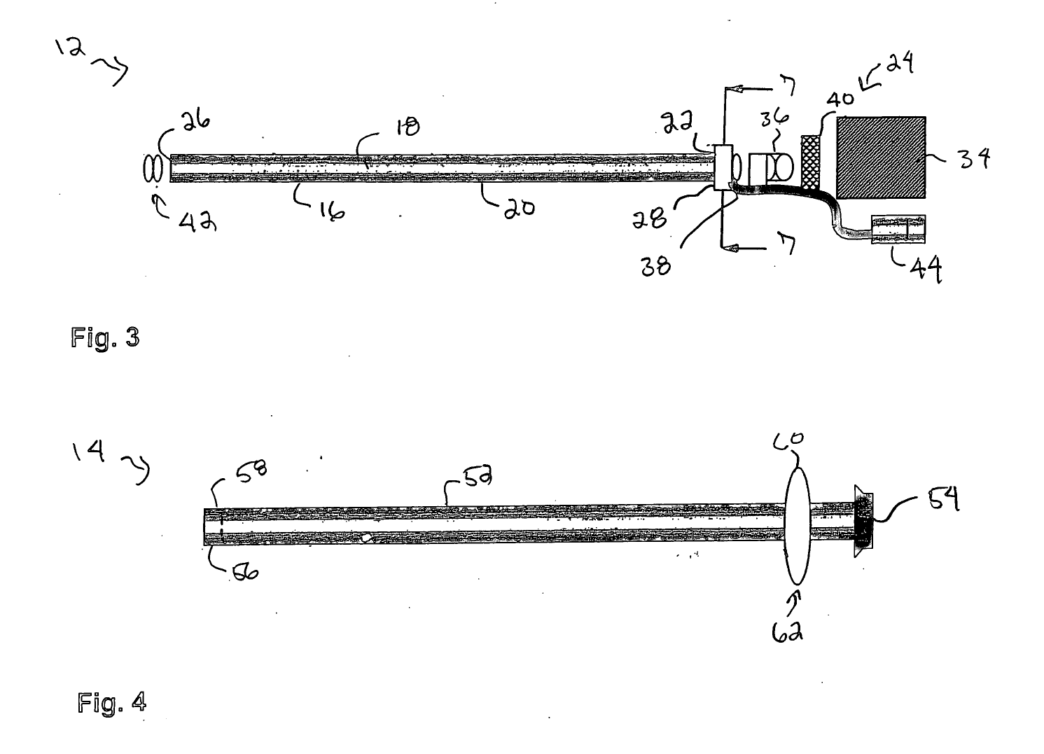

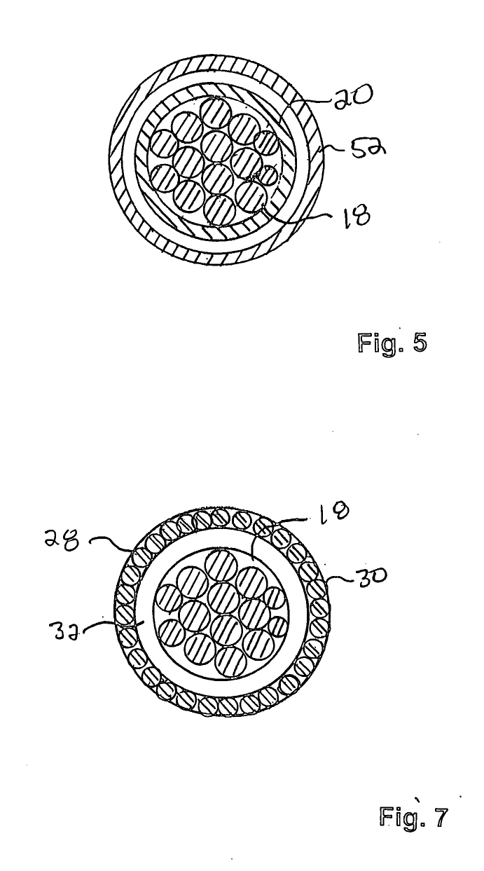

### DETAILED DESCRIPTION OF THE PREFERRED EMBODIMENT

With reference to the drawings wherein like numerals represent like parts throughout the several figures, a disposable illuminator endoscope in accordance with the present invention 10 includes a reusable endoscopic device 12 and a disposable illuminator 14.

The endoscopic device 12 includes an optical image pathway 16 comprising a coherent fiber optic imaging bundle or probe 18 for a flexible device 19 or a rod for a rigid device. Flexible optical probes 18 or rods having a 0.5-1 mm diameter may be used. The fiber optic bundle 18 is covered with a flexible metal sheath 20 that precludes infiltration of light into the fiber optic bundle 18. The rod of the rigid endoscope is also covered by a metal sheath, although this metal sheath need not be flexible. Since the remaining elements of the endoscopic device 10 are identical, only the flexible endoscope 19 will discussed in detail. The fiber optic bundle 18 extends from a proximal end portion 22, mounted to an operator assembly 24, to a distal (in body) end 26. A fiber optic annulus 28 surrounding the proximal end portion 22 of the fiber optic imaging bundle 18 includes a circular array of interface fiber optics 30 forming a light annulus 32 around the probe proximal end portion 22 (FIG. 7). The operator assembly 24 includes a CCD camera 34 (or CCD sensor array), and an imaging lens/imaging lens assembly 36 positioned between the proximal (out of body) end 38 of the fiber optic bundle 18 and the CCD camera 34. A focus adjustment 40 may be provided between the imaging lens/imaging lens assembly 36 and the CCD camera 34. The image is transmitted from the distal end 26 of the fiber optic bundle 18, through the imaging lens/imaging lens assembly 36 and the focus adjustment 40 to the CCD camera 34. One or more objective lens 42 may be positioned in front of the bundle distal end 26.

Conventional endoscopes image though an eyepiece that is designed for direct optical visual imaging with the eye. An optical device called an endo-coupler is required to interface with an electronic camera and the endoscope. Therefore, the optics are not optimized for camera imaging but for eye viewing. The subject disposable illuminator endoscope 10 is just the opposite, the disposable illuminator endoscope 10 is designed for optimum camera imaging with an optional eyepiece (not shown) that can be used in emergencies. The direct interface between the fiber optic imaging probe 18 and the CCD camera 34 provides superior optical transmission and lower costs, compared to conventional endoscopic devices.

The fiber optic annulus 28 is optically coupled to a fiber optic ferrule 44, which may be optically coupled to a light source 46. Preferably the light source 46 is a flashtube assembly 48 having a pulsed xenon flashtube 50 that emits a pulse of light of great intensity and broad spectrum but extremely short duration. For example, the flashtube 50 may emit a light pulse having the equivalent of 100,000 watts of light power, but lasting only 10 microseconds. Light provided by the flashtube assembly 48 is optically coupled to the disposable illuminator 14 by the fiber optic annulus 28.

The illuminator 14 is a flexible light path 52 that slides over the fiber optic imaging probe 18 to act as the illumination transmission path into the body. The proximal end 54 of the illuminator 14 interfaces with the circular array of the interface fiber optics 30 to optically couple the illuminator 14 to the light source 46. A sealing lens 56 mounted in the distal end 58 of the illuminator 14 provides an optically clear, fluid and gas tight seal that prevents contamination of the fiber optic imaging bundle 18 while allowing the illuminator 14 to transmit light into the body and the fiber optic imaging bundle 18 to receive images from within the body. A protective sleeve or condom 60 is mounted to the proximal end portion 62 of the illuminator 14. As shown in FIG. 2, the condom 60 is initially rolled-up when the fiber optic imaging probe 18 is inserted into the illuminator 14. The condom 60 is then be unrolled over the operator assembly 24 and cables to insure that the fiber optic imaging probe 18 remains in a sterile field. Upon completion of the procedure, the condom 60 is removed from around the operator assembly 24 and the illuminator 14 is discarded in an appropriate manner for medical waste. This enables the fiber optic imaging probe 18 to be used in multiple procedures without the necessity of re-sterilization. The illuminator 14 is pre-sterilized and packaged in blister packs, similar to conventional disposable needles.

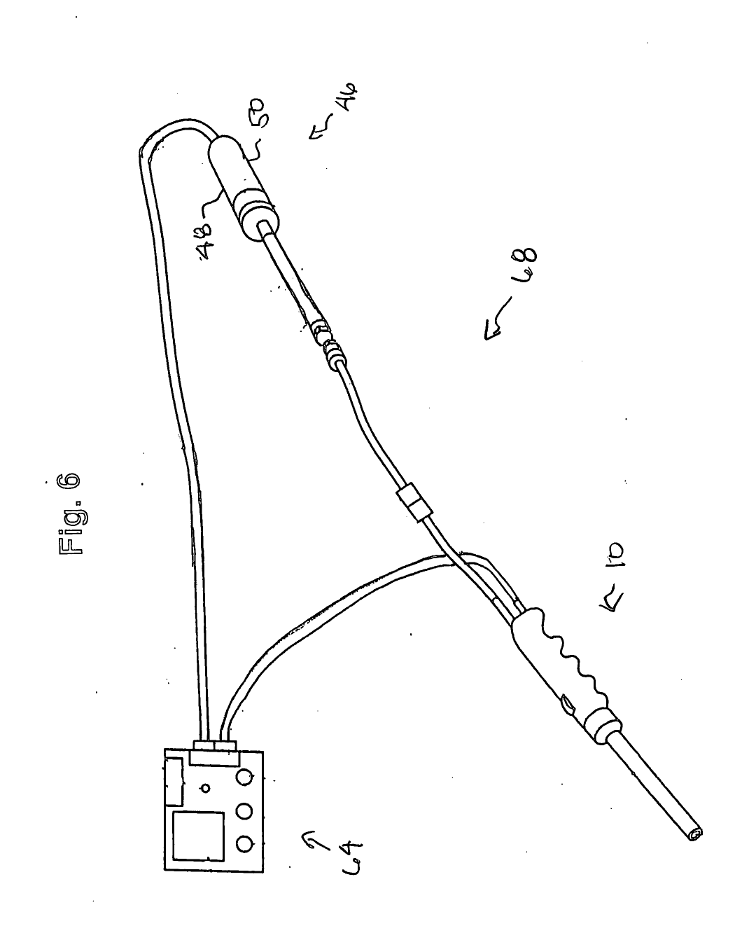

With reference to FIG. 6, the disposable illuminator endoscope 10 may be used with controls 64 that allow the surgeon to electronically increase the brightness of the image or to expand or contract the size of the image electronically. In addition, the surgeon retains the ability to control the magnification by moving the distal end 66 of the endoscope 10 closer or further away from the patient. In current operating rooms, the surgeon must have a second party to increase or decrease light levels as he inserts or removes the endoscope to affect magnification. In the subject system 68, these features are done automatically. The focus adjustment 40 on the operator assembly 24 allows the doctor to acquire the best focus for a given camera 34 and optical image pathway 16. The ergonomics of the disposable illuminator endoscope 10 is such that the operator assembly 24 is held like a knife with a handle grip 70 for reducing fatigue. To further facilitate use, controls are provided in the handle for brightness control, zoom control and rotation.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A disposable illuminator endoscope comprises:
a reusable endoscopic device including
an optical image pathway extending from a proximal end to a distal end,
an operator assembly mounted to the optical image pathway proximal end,
a fiber optic annulus surrounding a proximal end portion of the optical image pathway, and
a light source optically coupled to the fiber optic annulus; and
a disposable illuminator including
a light path extending from a proximal end to a distal end,
a sealing lens, mounted to the light path distal end, providing
an optically clear, fluid and gas tight seal, and
a protective sleeve having a distal end portion mounted to a proximal end portion of the light path;
wherein, the disposable illuminator is mountable on the reusable endoscopic device by inserting the optical image pathway into the light path and placing the protective sleeve around the operator assembly, whereby the distal end of the optical image pathway is disposed proximate to the sealing lens and the light path proximal end interfaces with fiber optic annulus to optically couple the disposable illuminator to the light source, and the disposable illuminator is removable from the reusable endoscopic device by withdrawing the optical image pathway and the operator assembly from the light path and the protective sleeve, respectively.

2. The disposable illuminator endoscope of claim 1 wherein the optical image pathway comprises a flexible, coherent fiber optic imaging bundle or probe, and the light path is composed of a flexible material.

3. The disposable illuminator endoscope of claim 1 wherein the disposable illuminator is pre-sterilized and packaged in a sterile container.

4. The disposable illuminator endoscope of claim 1 wherein the optical image pathway is covered with a metal sheath that precludes infiltration of light into the optical image pathway.

5. The disposable illuminator endoscope of claim 4 wherein the metal sheath is flexible.

6. The disposable illuminator endoscope of claim 1 wherein the fiber optic annulus includes a circular array of interface fiber optics forming a light annulus around the optical image pathway proximal end portion.

7. The disposable illuminator endoscope of claim 1 wherein the operator assembly includes
a CCD camera or CCD sensor array and
an imaging lens assembly positioned between the proximal end of the optical image pathway and the CCD camera or CCD sensor array.

8. The disposable illuminator endoscope of claim 7 wherein the operator assembly also includes a focus adjustment disposed between the imaging lens assembly and the CCD camera or CCD sensor array.

9. The disposable illuminator endoscope of claim 8 wherein the operator assembly also includes at least one objective lens.

10. The disposable illuminator endoscope of claim 1 wherein the reusable endoscopic device also includes a fiber optic ferrule coupling the fiber optic annulus to the light source.

11. The disposable illuminator endoscope of claim 1 wherein the light source is a flashtube assembly having a pulsed xenon flashtube.

12. A disposable illuminator endoscope comprises:
a reusable endoscopic device including
an optical image pathway extending from a proximal end to a distal end, the optical image pathway comprises a flexible, coherent fiber optic imaging bundle or probe,
a flexible metal sheath covering the optical image pathway,
an operator assembly mounted to the optical image pathway proximal end,
a circular array of fiber optics forming a light annulus around a proximal end portion of the optical image pathway, and
a light source optically coupled to the fiber optic annulus; and
a disposable illuminator including
a flexible light path extending from a proximal end to a distal end,
a sealing lens, mounted to the light path distal end, providing an optically clear, fluid and gas tight seal, and
a protective sleeve having a distal end portion mounted to a proximal end portion of the light path;
wherein, the disposable illuminator is mountable on the reusable endoscopic device by inserting the optical image pathway into the light path and placing the protective sleeve around the operator assembly, whereby the distal end of the optical image pathway is disposed proximate to the sealing lens and the light path proximal end interfaces with fiber optic annulus to optically couple the disposable illuminator to the light source, and the disposable illuminator is removable from the reusable endoscopic device by withdrawing the optical image pathway and the operator assembly from the light path and the protective sleeve, respectively.
